(19) United States
(12) Patent Application Publication
(10) Pub. No.: US 2026/0108515 A1
(43) Pub. Date: Apr. 23, 2026

(54) A RAPIDLY DISINTEGRATING TABLET OF ROSUVASTATIN AND ITS PROCESS OF PREPARATION

(71) Applicant: NOVUMGEN LIMITED, London (GB)

(72) Inventor: Kamleshkumar Patel, London (GB)

(21) Appl. No.: 19/116,269

(22) PCT Filed: Sep. 15, 2023
(86) PCT No.: PCT/GB2023/052392

§ 371 Date: Mar. 27, 2025

(30) Foreign Application Priority Data
Sep. 28, 2022 (GB) 2214218.6

(51) Int. Cl. A61K 31/505; A61K 9/00; A61K 47/12; A61K 47/26; A61K 47/36; A61K 47/38

(52) U.S. Cl. CPC A61K 31/505; A61K 9/0056; A61K 47/12; A61K 47/26; A61K 47/36; A61K 47/38

(57) ABSTRACT

The present invention relates to a rapidly disintegrating tablet that can disintegrate in the buccal cavity upon contact with saliva in less than 1 minute, preferably in less than 40 seconds. The tablet comprises rosuvastatin or pharmaceutically acceptable salts thereof, as well as a combination of excipients that include at least one disintegrating agent and at least one diluent agent. The present invention is based on enhancing the disintegration and dissolution of the drug for oral administration.

### FIELD OF THE INVENTION

The present invention relates to a pharmaceutical composition of rosuvastatin. The present invention relates to a rapidly disintegrating tablet of rosuvastatin or pharmaceutically acceptable salts thereof for oral administration. The present invention also relates to the process of the preparation of the same.

### BACKGROUND OF THE INVENTION

Rosuvastatin was first disclosed in the U.S. Pat. No. 5,260,440. Rosuvastatin is a lipid-lowering drug that belongs to a class of medications called HMG-CoA reductase inhibitors and is used to lower low-density lipoprotein (LDL) cholesterol levels and raise high-density lipoprotein (HDL) cholesterol levels. Additionally, it lowers triglyceride levels (TG) and improves cholesterol levels, which can help prevent arteries from being blocked. These blockages may result in severe problems, including heart attacks or strokes.

Rosuvastatin competitively inhibits the enzyme hydroxymethylglutaryl coenzyme A (HMG-CoA) reductase that is responsible for cholesterol synthesis in the body, which catalyses the conversion of HMG-CoA to mevalonic acid, which is the third step in a series of metabolic reactions involved in the production of several compounds involved in lipid metabolism and transport, including cholesterol, low-density lipoprotein, and very low-density lipoprotein. The use of statin medications is considered standard practice for people who have a moderate to high risk of developing cardiovascular disease, such as those with Type 2 diabetes. Rosuvastatin is effective in the treatment of hypercholesterolemia, hyperlipoproteinemia, hyperlipidaemia, and mixed dyslipidaemia, hypertriglyceridemia, myocardial infraction, atherosclerosis, and stroke.

The IUPAC name of rosuvastatin is (E,3R,5S)-7-[4-(4-fluorophenyl)-2-[methyl(methylsulfonyl)amino]-6-propan-2-ylpyrimidin-5-yl]-3,5-dihydroxyhept-6-enoic acid.

Rosuvastatin is used for medical purposes in the form of its pharmaceutically acceptable salts, mainly as the calcium salt or as the zinc salts. Rosuvastatin belongs to the biopharmaceutics classification system (BCS) class II, which has high permeability and low solubility. The primary cause of new drug development failure is due to the poor solubility of the drug candidate, which frequently results in low bioavailability and eventually therapeutic failure. Drug permeability is recognised as the second crucial factor influencing oral bioavailability. The solubility of drug is often intrinsically related to drug particle size; as a particle becomes smaller, the surface area to volume ratio increases. The larger surface area allows greater interaction with the solvent, which causes an increase in solubility.

In the US and Europe, rosuvastatin is marketed as capsules and tablets in strengths of 5 mg, 10 mg, 20 mg, and 40 mg. Its unpleasant, strong bitter taste after oral administration presents a special challenge in the development of oral pharmaceutical formulations.

The currently available solid dosage form of rosuvastatin has relatively lengthy onset of action. Tablets and capsules are particularly difficult to swallow for aged and physically disabled patients since they taste unpleasant and are difficult to swallow even when taken with liquid. Additionally, some patients don't take their medication on a regular basis, which has a significant impact on the effectiveness of the treatment and poor patient compliance. Rapidly disintegrating tablets with good mouth feeling may help in strengthens the psychological belief on medication. The rapidly disintegrating tablet is made with the goal of achieving rapid disintegration of the tablet when it comes into contact with saliva. Improved safety by preventing chocking or obstruction as in the case of conventional dosage forms during swallowing.

Rosuvastatin has a plasma half-life of 18-24 hours. Greater low-density lipoprotein—Cholesterol reduction can be obtained in severe hypercholesterolaemia, partly due to its longer persistence in the plasma. In patients with raised triglyceride levels, rosuvastatin raises high-density lipoprotein—Cholesterol by 15-20%. Rosuvastatin is metabolized primarily by CYP3A4. Inhibitors and inducers of this isoenzyme, respectively, increase and decrease statin blood levels.

U.S. Pat. No. 6,316,460 discloses an oral tablet containing rosuvastatin as the active ingredient, one or more fillers, one or more binders, one or more disintegrants, one or more lubricants, and a tribasic phosphate Salt. The dry blending and wet granulation processes are used to produce the tablet. The active ingredient and additional excipients are blended together during dry blending, and the mixture is then passed through a mesh. A lubricant is gradually added to the mixture with continuous blending until a homogeneous mixture is formed. After that, the mixture is compressed into a tablet. In the wet granulation method, the rosuvastatin, the tribasic phosphate salt, povidone, microcrystalline cellulose, lactose, sodium Starch glycolate, butylated hydroxytoluene, magnesium stearate and other additional excipients are blended together by using a granulator, and the powder blend is granulated with a small volume of purified water. The granules are dried and passed through a mill. The remainder of the disintegrant and a lubricant are added to the milled granulation and, after blending, the resultant homogeneous mixture is compressed into tablets. A tablet coating may be applied by Spray-coating with a water-based film coating formulation. This innovation has several drawbacks as coating is a tedious and time-consuming technique that increases formulation costs. Tablets are difficult to swallow by children, sick, unconscious, and seriously ill patients. The pharmacodynamics properties of drug formulations may be affected by tablet coating, and the process occasionally leads to coating defects such as chipping and cracking etc. and the process for coating tablets remains complicated.

WO 2021/064306 discloses an effervescent formulation comprising of rosuvastatin and/or a pharmaceutically acceptable salt thereof for oral administration. The effervescent tablet comprises at least one organic salt, at least one effervescent acid and base, and at least one sterol absorption inhibitor. The present innovation has limitations that include a trained individual, the requirement for more expensive excipients, a difficult manufacturing process, specialised packaging materials, and the need for larger tablets.

W02011/018185 discloses a compression-coated tablet of rosuvastatin calcium. The process of the invention comprises compressing a tablet blend containing rosuvastatin calcium, dibasic calcium phosphate, and at least one pharmaceutically acceptable excipient to form a compressed core. Furthermore, a moisture barrier layer made of polyvinyl alcohol is spray-coated and pan-coated onto the compressed core. This innovation has drawbacks, such as compression coated tablets produce a significant increase in the size and weight of the core tablet, and care must be taken during the formulation and processing of the coating layer due to the complexity of the mechanism used in the compressing equipment.

US 2009/0297599 discloses a film coated tablet of rosuvastatin calcium that comprises at least one stabilising additive and at least one pharmaceutically acceptable excipient. The process for preparing the pharmaceutical composition comprises sieving the active ingredient and stabilising additives, which is then homogenised with the diluent, binder, and disintegrant. Then, lubricant is added, and the mixture is blended before being pressed into tablets. Then the tablets are film-coated with an aqueous solution of Opadry II.

### SUMMARY OF THE INVENTION

In accordance with the present invention, the rapidly disintegrating tablet for oral administration is prepared. The rapidly disintegrating tablet comprises rosuvastatin calcium, at least one disintegrant, and at least one diluent. The rapidly disintegrating tablet further comprises one or more pharmaceutical acceptable excipient.

Another embodiment of the invention involves a process for preparing the rapidly disintegrating tablet for oral administration. The rapidly disintegrating tablet is prepared by the direct compression method.

Another embodiment of the invention can effectively treat hyperlipidaemia, mixed dyslipidaemia, hypercholesterolemia, hyperlipoproteinemia, hypertriglyceridemia, myocardial infraction, atherosclerosis, and stroke.

### OBJECTS OF THE INVENTION

The primary object of the present invention is to provide a rapidly disintegrating tablet of rosuvastatin or pharmaceutically acceptable salts thereof.

Another object of the present invention is to enhance the disintegration and dissolution of the drug for oral administration.

Still another object of the present invention is to provide a rapidly disintegrating tablet of rosuvastatin or pharmaceutically acceptable salts thereof, suitable for oral administration by the direct compression method.

Still another object of the present invention is to provide taste masking properties and a pleasant palatability for children, elderly, or unconscious patients, and thus patient compliance is improved.

Yet another object of the present invention is to provide a stable rapidly disintegrating tablet of rosuvastatin or pharmaceutically acceptable salts thereof.

### DETAILED DESCRIPTION OF THE INVENTION

The rapidly disintegrating tablet of rosuvastatin suitable for oral administration is the invention as further described herein.

Furthermore, the term “Rapidly disintegrating tablet”, as used in the present invention, refers to uncoated tablet that are intended to be placed in the mouth and disintegrate rapidly before being swallowed. Rapidly disintegrating tablets are also known as orally disintegrating tablets, orodispersible tablets, or orally dispersible tablets. The rapidly disintegrating tablet can be administered to any patient having difficulty swallowing.

Drug bioavailability depends on drug absorption. In the case of poorly water-soluble drugs like rosuvastatin, the bioavailability of the drug depends mainly on its dissolution, which is affected by drug dosage form disintegration. As a result, a rapidly disintegrating tablet formulation could aid in drug dissolution and absorption.

The preferred embodiment of the invention is a rapidly disintegrating tablet suitable for oral administration comprising rosuvastatin, at least one disintegrant, and at least one diluent. When in contact with saliva, this tablet disintegrates, release the active ingredient and ensuring maximum drug bioavailability compared to conventional dose forms. In one embodiment, the rosuvastatin calcium is present in the range of about 1% w/w to about 8% w/w, preferably in the range of about 2% w/w to about 7% w/w and is administered as an oral solid dosage form.

The term “about”, as and when used in this specification, means ±10% of the mentioned value.

The present invention is a rapidly disintegrating tablet for oral administration characterized by physicochemical properties suitable for a tablet formulation manufactured by the direct compression method, by an adequate release rate of the active ingredient, and storage stability achieved by employing excipients practically devoid of the tendency to interact with the active ingredient and possessing good compressibility properties. The term “D90 diameter” refers to the particle size that accounts for 90% of the volume of rosuvastatin particles. The D90 particle size of rosuvastatin or a pharmaceutically acceptable salt thereof, can be in the range of 50 to 300 μm, more preferably in the range of 120 to 200 μm. The D50 is the corresponding particle size when the cumulative percentage reaches 50%. D50 is also known as the “mass median diameter” as it divides the sample equally by mass, The D50 particle size of rosuvastatin or a pharmaceutically acceptable salt thereof, can be in the range of 15 to 90 μm, more preferably in the range of 20 to 60 μm. D10 represents the particle diameter corresponding to 10% cumulative undersize particle size distribution. The D10 particle size of rosuvastatin or a pharmaceutically acceptable salt thereof, can be in the range of 1 to 20 μm, more preferably in the range of 2 to 10 μm.

In one embodiment, suitable diluent for the present invention is selected from dextrates, microcrystalline cellulose, dextrose, fructose, Sorbitol, pregelatinized starch, starch, xylitol, sucrose, maltodextrin, maltose, mannitol and combinations thereof. In the present invention combination of the microcrystalline cellulose and mannitol are preferred as diluent in range of about 10% w/w to about 90% w/w, preferably in the range from about 15% w/w to about 85% w/w. microcrystalline cellulose is present in the range from about 10% w/w to about 75% w/w, preferably from about 15% w/w to about 60% w/w, and mannitol is present in the range from about 20% w/w to about 90% w/w, preferably from about 50% w/w to about 85% w/w. Mannitol has negative heat of solution which provides ‘mouth feel’ effect. Therefore, it is used in combination with microcrystalline cellulose in comparison with other diluents in the present invention.

In one embodiment, a suitable disintegrant for the present invention is selected from methylcellulose, alginic acid, carboxymethylcellulose calcium, guar gum, carboxymethylcellulose Sodium, polacrilin potassium, croscarmellose sodium, poloxamer, sodium starch glycolate and sodium alginate. Further, the disintegrant can be single or any combination thereof. Croscarmellose sodium is the preferred disintegrant for the present invention in the range from about 0.5% w/w to about 10% w/w, preferably in the range from about 1% w/w to about 7.5% w/w. croscarmellose sodium is an internally cross-linked with sodium carboxymethylcellulose. Through the mechanisms of swelling, recovering elastic energy, and capillary action, croscarmellose sodium speeds up the wicking process and ultimately reducing the disintegration time. Further, it has the tendency to absorb water rapidly, so it swells significantly. Therefore, this rapid absorption of water by croscarmellose sodium result in a significant increase in the volume of granules, resulting in rapid and uniform disintegration. Therefore, croscarmellose sodium is the disintegrant of choice for rapidly disintegrating tablet of rosuvastatin calcium.

As per another embodiment, the lubricant is selected from boric acid, magnesium stearate, Sodium Stearyl fumarate, micronized polyoxyethyleneglycol, leucine, sodium benzoate, sodium acetate, sodium lauryl sulphate, steric acid, sodium stearate, sodium oleate, calcium stearate, waxes or mixtures thereof. Magnesium stearate is preferred as a lubricant for the present invention and is present in the range from about 0.25% w/w to about 5% w/w, preferably in the range from about 0.5% w/w to about 4% w/w.

As per another embodiment, sweetener is in range from 0.05 to 5% w/w, preferably from about 0.1 to 2% w/w. In one embodiment, a suitable sweetener for the present invention is selected from cyclamate, acesulfame potassium, neo hesperidin dihydrochalcone, monoammonium glycyrrhizinate, saccharin sodium, sucralose, saccharin, aspartame or mixtures thereof. Sucralose is used as sweetener for the rapidly disintegrating tablet of the present invention.

As per another embodiment, the rapidly disintegrating tablet further comprising a flavouring agent. Flavouring agents may be, for example, menthol, mint powder, vanillin, or orange flavour. The rapidly disintegrating tablet of the present invention can be prepared in the presence of orange flavour present in the range from about 0.25% w/w to about 5% w/w, preferably in the range from about 0.5% w/w to about 4% w/w.

As per another embodiment, the solid pharmaceutical composition of the present invention remains stable at different temperature conditions.

In the preferred embodiment of the present invention, is to provide a rapidly disintegrating tablet suitable for oral administration comprising rosuvastatin, at least one disintegrant, and at least one diluent. In addition, the rapidly disintegrating tablet further comprises at least one pharmaceutically acceptable excipient selected from sweetener, flavouring agent, and lubricant.

Another embodiment of the present invention is the use of the direct compression method for the preparation of rapidly disintegrating tablet containing rosuvastatin, which is one of the most economical methods.

As per another embodiment, the disintegrating time of the rosuvastatin rapidly disintegrating tablet is less than 1 minute, preferably less than 40 seconds.

As per another embodiment, 85% of the rosuvastatin is released in 30 minutes, preferably more than 90% is released within 20 minutes.

As per another embodiment, the direct compression method is used to prepare the rapidly disintegrating tablet. Rosuvastatin calcium and microcrystalline cellulose 102 are sieved together through a 40 # sieve. Mannitol and croscarmellose sodium are sieved separately through a 40 # sieve. Sucralose, orange flavor, and magnesium stearate are sieved separately through a 60 # sieve. In a blender, mannitol, rosuvastatin calcium and microcrystalline cellulose 102 are blended together. Previously sifted croscarmellose sodium, sucralose, and orange flavor is added to the above blend to prepare the final mixture, except magnesium stearate, and mixed for 15 minutes. Magnesium stearate is added to the final mixture and properly mixed for 5 minutes. Finally, the lubricated mixture is compressed to form tablets.

The invention is further illustrated by the following examples, which are by no means intended to limit the scope of the invention but are given by way of illustration.

### Example 1

| Ingredients | % w/w |
|---|---|
| Rosuvastatin (Rosuvastatin as equivalent | 5.00 |
| to Rosuvastatin Calcium) | |
| Microcrystalline cellulose-101 | 18.00 |
| Mannitol | 72.75 |
| Sodium starch glycolate | 2.00 |
| Sucralose | 0.25 |
| Orange flavour | 1.00 |
| Magnesium Stearate | 1.00 |
| Total | 100.00 |

### Manufacturing Process:

Rosuvastatin calcium is mixed with approximately 1/10 quantity of mannitol. The prepared mixture is sieved through a 40 # sieve. Manitol, microcrystalline cellulose-101, and sodium starch glycolate are sieved separately through a 40 # sieve. Sucralose, orange flavor, and magnesium stearate are sieved separately through a 60 # sieve. Mannitol, microcrystalline cellulose 101, sodium starch glycolate, sucralose, orange flavor and rosuvastatin calcium are blended together in a blender and mixed for 20 minutes. Magnesium stearate is added to the final mixture and properly mixed for 5 minutes.

The prepared blended mixture shows the characteristics mentioned in the table below:

| Test Parameters of blend | Result |
|---|---|
| Bulk Density (gm/ml) | 0.552 |
| Tap Density (gm/ml) | 0.747 |
| Carr's Index (%) | 26.10 (poor) |
| Hausner's Ratio | 1.35 |

### Example 2

| Ingredients | % w/w |
|---|---|
| Rosuvastatin (Rosuvastatin as equivalent | 5.00 |
| to Rosuvastatin Calcium) | |
| Microcrystalline cellulose-102 | 18.00 |
| Mannitol | 72.75 |
| Sodium starch glycolate | 2.00 |
| Sucralose | 0.25 |
| Orange flavour | 1.00 |
| Magnesium Stearate | 1.00 |
| Total | 100.00 |

### Manufacturing Process:

Rosuvastatin calcium is mixed with approximately 1/10 quantity of mannitol. The prepared mixture is sieved through a 40 # sieve. Mannitol, microcrystalline cellulose-102, and sodium starch glycolate are sieved separately through a 40 # sieve. Sucralose, orange flavor, and magnesium stearate are sieved separately through a 60 # sieve. Mannitol, microcrystalline cellulose 102, sodium starch glycolate, sucralose, and orange flavor are blended together in a blender and mixed for 20 minutes. Magnesium stearate is added to the final mixture and properly mixed for 5 minutes. Finally, the lubricated mixture is compressed to form tablets.

| Test Parameters of blend | Result |
|---|---|
| Bulk Density (gm/ml) | 0.674 |
| Tap Density (gm/ml) | 0.845 |
| Carr's Index (%) | 20.22 |
| Hausner's Ratio | 1.25 |

The tablets present the characteristics mentioned in the table below:

| Test Parameters | Result |
|---|---|
| Weight (mg) | 201.4 |
| Hardness (N) | 49 |
| Friability (%) | 0.12 |
| Buccal disintegration time (seconds) | 3 min 45 sec |

### Example 3

| Ingredients | % w/w |
|---|---|
| Rosuvastatin (Rosuvastatin as equivalent | 5.00 |
| to Rosuvastatin Calcium) | |
| Microcrystalline cellulose-102 | 18.00 |
| Mannitol | 70.75 |
| Sodium starch glycolate | 4.00 |
| Sucralose | 0.25 |
| Orange flavour | 1.00 |
| Magnesium Stearate | 1.00 |
| Total | 100.00 |

### Manufacturing Process:

Rosuvastatin calcium is mixed with approximately 1/10 quantity of mannitol. The prepared mixture is sieved through a 40 # sieve. Mannitol microcrystalline cellulose-102, and sodium starch glycolate are sieved separately through a 40 # sieve. Sucralose, orange flavor, and magnesium stearate are sieved separately through a 60 # sieve. Mannitol, microcrystalline cellulose 102 and rosuvastatin calcium are blended together in a blender. Previously sifted sodium starch glycolate, sucralose, and orange flavor is added to the above blend to prepare the final mixture, except magnesium stearate, and mixed for 15 minutes. Magnesium stearate is added to the final mixture and properly mixed for 5 minutes. Finally, the lubricated mixture is compressed to form tablets.

| Test Parameters of blend | Result |
|---|---|
| Bulk Density (gm/ml) | 0.665 |
| Tap Density (gm/ml) | 0.824 |
| Carr's Index (%) | 19.35 |
| Hausner's Ratio | 1.24 |

The tablets present the characteristics mentioned in the table below:

| Test Parameters | Result |
|---|---|
| Weight (mg) | 199.8 |
| Hardness (N) | 43 |
| Friability (%) | 0.15 |
| Buccal disintegration time (seconds) | 3 min 10 sec |

### Example 4

| Ingredients | % w/w |
|---|---|
| Rosuvastatin (Rosuvastatin as equivalent | 5.00 |
| to Rosuvastatin Calcium) | |
| Microcrystalline cellulose-102 | 18.00 |
| Mannitol | 70.75 |
| Croscarmellose sodium | 4.00 |
| Sucralose | 0.25 |
| Orange flavour | 1.00 |
| Magnesium Stearate | 1.00 |
| Total | 100.00 |

### Manufacturing Process:

Rosuvastatin calcium is mixed with approximately 1/10 quantity of mannitol. The prepared mixture is sieved through a 40 # sieve. Mannitol, microcrystalline cellulose-102, and croscarmellose sodium are sieved separately through a 40 # sieve. Sucralose, orange flavor, and magnesium stearate are sieved separately through a 60 # sieve. Mannitol, microcrystalline cellulose 102, and rosuvastatin calcium are blended together in a blender. Previously sifted croscarmellose sodium, sucralose, and orange flavor is added to the above blend to prepare the final mixture, except magnesium stearate, and mixed for 15 minutes. Magnesium stearate is added to the final mixture and properly mixed for 5 minutes. Finally, the lubricated mixture is compressed to form tablets.

| Test Parameters of blend | Result |
|---|---|
| Bulk Density (gm/ml) | 0.681 |
| Tap Density (gm/ml) | 0.841 |
| Carr's Index (%) | 19.03 |
| Hausner's Ratio | 1.24 |

The tablets present the characteristics mentioned in the table below:

| Test Parameters | Result |
|---|---|
| Weight (mg) | 199.7 |
| Hardness (N) | 44 |
| Friability (%) | 0.11 |
| Buccal disintegration time (seconds) | 35 |

### Example 5

| Ingredients | % w/w |
|---|---|
| Rosuvastatin (Rosuvastatin as equivalent | 5.00 |
| to Rosuvastatin Calcium) | |
| Microcrystalline cellulose-102 | 18.00 |
| Mannitol | 70.35 |
| Croscarmellose sodium | 4.00 |
| Sucralose | 0.65 |
| Orange flavour | 1.00 |
| Magnesium Stearate | 1.00 |
| Total | 100.00 |

### Manufacturing Process:

Rosuvastatin calcium is mixed with approximately 1/10 quantity of mannitol. The prepared mixture is sieved through a 40 # sieve. Mannitol, microcrystalline cellulose-102, and croscarmellose sodium are sieved separately through a 40 # sieve. Sucralose, orange flavor, and Magnesium stearate are sieved separately through a 60 # sieve. Mannitol, microcrystalline cellulose 102 and rosuvastatin calcium are blended together in a blender, Previously sifted Croscarmellose sodium, sucralose, and orange flavor is added to the above blend to prepare the final mixture, except magnesium stearate, and mixed for 15 minutes. Magnesium stearate is added to the final mixture and properly mixed for 5 minutes. Finally, the lubricated mixture is compressed to form tablets.

| Test Parameters of blend | Result |
|---|---|
| Bulk Density (gm/ml) | 0.680 |
| Tap Density (gm/ml) | 0.843 |
| Carr's Index (%) | 19.35 |
| Hausner's Ratio | 1.24 |

The tablets present the characteristics mentioned in the table below:

| Test Parameters | Result |
|---|---|
| Weight (mg) | 201.4 |
| Hardness (N) | 36 |
| Friability (%) | 0.14 |
| Buccal disintegration time (seconds) | 42 |

### Example 6

| | | Quantity | Quantity |
|---|---|---|---|
| Ingredients | % w/w | (5 mg/tablet) | (10 mg/tablet) |
| Rosuvastatin (Rosuvastatin | 5.00 | 5.00 | 10.00 |
| as equivalent to | | | |
| Rosuvastatin Calcium) | | | |
| Microcrystalline | 18.00 | 18.00 | 36.00 |
| cellulose-102 | | | |
| Mannitol | 70.35 | 70.35 | 140.70 |
| Croscarmellose sodium | 4.00 | 4.00 | 8.00 |
| Sucralose | 0.65 | 0.65 | 1.30 |
| Orange flavour | 1.00 | 1.00 | 2.00 |
| Magnesium Stearate | 1.00 | 1.00 | 2.00 |
| Total | 100.00 | 100.00 | 200.00 |

### Manufacturing Process:

Rosuvastatin calcium and microcrystalline cellulose 102 are sieved together through a 40 # sieve. Mannitol and croscarmellose sodium are sieved separately through a 40 # sieve. Sucralose, orange flavor, and magnesium stearate are sieved separately through a 60 # sieve. In a blender mannitol, rosuvastatin calcium and microcrystalline cellulose 102 are blended together. Previously sifted Croscarmellose sodium, sucralose, and orange flavor is added to the above blend to prepare the final mixture, except magnesium stearate, and mixed for 15 minutes. Magnesium stearate is added to the final mixture and properly mixed for 5 minutes. Finally, the lubricated mixture is compressed to form tablets.

| Test Parameters | Result |
|---|---|
| Bulk Density (gm/ml) | 0.6614 |
| Tap Density (gm/ml) | 0.8378 |
| Carr's Index (%) | 21.05 |
| Hausner's Ratio | 1.267 |

### Example 7: The Dissolution Profile of the Tablet Prepared According the Example 6

A dissolution profile is made with the rapidly disintegrating tablet of example 6

The conditions of dissolution are the following:
Apparatus: USP type II (paddle)
Rate of rotation: 50 rpm
Volume: 500 ml
Temperature: 37° C.±0.5° C.
Detection: Direct UV spectrophotometry at 242 nm
Dissolution medium: at pH 6.6 citrate buffer

### Dissolution Profile of Rosuvastatin 5 mg Rapidly Disintegrating Tablet

Example 6 (Cumulative % Drug Release)

pH 6.6 Citrate Buffer

| Time (min) | % Release | % Relative Standard Deviation |
|---|---|---|
| 10 | 95.9 | 2.1 |
| 15 | 96.4 | 2.8 |
| 20 | 97.0 | 2.7 |
| 30 | 99.4 | 3.0 |
| 45 | 97.1 | 2.8 |
| 60 | 96.8 | 2.3 |

### Dissolution Profile of Rosuvastatin 10 mg Rapidly Disintegrating Tablet

Example 6 (Cumulative % Drug Release)

pH 6.6 Citrate Buffer

| Time (min) | % Release | % Relative Standard Deviation |
|---|---|---|
| 10 | 96.5 | 2.7 |
| 15 | 97.3 | 1.8 |
| 20 | 98.0 | 2.1 |
| 30 | 99.0 | 2.5 |
| 45 | 99.1 | 2.6 |
| 60 | 99.5 | 2.6 |

Example 8: The tablet prepared according to example 6 were subjected to stability study 40° C.±2° C./75% RH±5% RH for 1 month. Results are tabulated below.

### Rosuvastatin 5 mg Rapidly Disintegrating Tablet

| | | | Exp. 6 |
|---|---|---|---|
| | | | (After 1 |
| | | Exp. 6 | month at |
| Test | Specification | (Initial) | 40° C. ± 2° C.) |
| Average | 100.0 mg ± 7.5% | 101.0 | 99.50 |
| weight | (92.5 mg-107.5 mg) | | |
| Hardness | 15-65N (Target 40N) | 42N | 33N |
| Friability | NMT 1.0% | 0.15% | 0.11% |
| Disintegration | NMT 3 Minute | 20 sec | 25 sec |
| time | | | |
| Assay | 95.0-105.0% of label | 100.1 | 99.8% |
| | claim. | | |
| Dissolution | NLT 75% (Q) labelled | 96.4 | 90.3 |
| | amount of Rosuvastatin | | |
| | should be dissolve in | | |
| | 15 minutes. | | |

### Rosuvastatin 10 Mg Rapidly Disintegrating Tablet

| | | | Exp. 6 |
|---|---|---|---|
| | | | (After 1 |
| | | | month at |
| | | Exp. 6 | 40° C. ± |
| Test | Specification | (Initial) | 2° C.) |
| Average | 200.0 mg ± 7.5% | 202.10 | 200.70 |
| weight | (185.0 mg-215.0 mg) | | |
| Hardness | 15-65N (Target 40N) | 33N | 50N |
| Friability | NMT 1.0% | 0.13% | 0.16% |
| Disintegration | NMT 3 Minute | 23 sec | 13 sec |
| time | | | |
| Assay | 95.0-105.0% of label claim. | 99.9 | 98.0 |
| Dissolution | NLT 75% (Q) labelled | 97.3 | 97.9 |
| | amount of Rosuvastatin | | |
| | should be dissolve in 15 | | |
| | minutes. | | |

1. A rapidly disintegrating tablet suitable for oral administration comprising rosuvastatin or pharmaceutically acceptable salts thereof, at least one disintegrant in the range of about 0.5% w/w to about 10% w/w, preferably in the range of about 1% w/w to about 7.5% w/w and at least one diluent.

2. The rapidly disintegrating tablet according to claim 1, wherein the therapeutically effective amount of rosuvastatin or pharmaceutically acceptable salts thereof, is present in an amount ranging from about 1% w/w to about 8% w/w, preferably in the range from about 2% w/w to about 7% w/w.

3. The rapidly disintegrating tablet according to claim 1, wherein the disintegrant is selected from methylcellulose, alginic acid, carboxymethylcellulose calcium, carboxymethylcellulose sodium, polacrilin potassium, croscarmellose sodium, guar gum, poloxamer, sodium starch glycolate, and sodium alginate or any combination thereof.

4. The rapidly disintegrating tablet according to claim 3, wherein the disintegrant is Croscarmellose sodium.

5. The rapidly disintegrating tablet according to claim 1, wherein the diluent is selected from dextrates, microcrystalline cellulose, dextrose, fructose, sorbitol, pregelatinized starch, starch, xylitol, sucrose, maltodextrin, maltose, mannitol, or combinations thereof.

6. The rapidly disintegrating tablet according to claim 5, wherein the diluent is a combination of microcrystalline cellulose and mannitol present in the range of about 10% w/w to about 90% w/w, preferably in the range of about 15% w/w to about 85% w/w.

7. The rapidly disintegrating tablet according to claim 1, further comprising at least one pharmaceutically acceptable excipient selected from lubricant, sweetener, and flavoring agent.

8. The rapidly disintegrating tablet according to claim 7, wherein the lubricant is selected from the group consisting of boric acid, magnesium stearate, sodium Stearyl fumarate, micronized polyoxyethyleneglycol, leucine, sodium benzoate, sodium acetate, sodium lauryl sulphate, steric acid, sodium stearate, sodium oleate, calcium stearate, and combinations thereof.

9. The rapidly disintegrating tablet according to claim 7, wherein the sweetener is selected from the group consisting of cyclamate, acesulfame potassium, neo hesperidin dihydrochalcone, monoammonium glycyrrhizinate, saccharin sodium, sucralose, saccharin, aspartame, and combinations thereof.

10. The rapidly disintegrating tablet according to claim 7, wherein the flavoring agent is selected from menthol, mint powder, vanillin, or orange flavor.

11. The rapidly disintegrating tablet according to claim 7, further comprising magnesium stearate, sucralose, and orange flavor.

12. The rapidly disintegrating tablet according to claim 1, wherein the D90 particle size of rosuvastatin or pharmaceutically acceptable salt thereof, is in the range of about 50 to 300 μm, more preferably in the range of about 120 to 200 μm.

13. The rapidly disintegrating tablet according to claim 1, wherein the D50 particle size of rosuvastatin or pharmaceutically acceptable salt thereof, is in the range of about 15 to 90 μm, more preferably in the range of about 20 to 60 μm.

14. The rapidly disintegrating tablet according to claim 1, wherein the D10 particle size of rosuvastatin or pharmaceutically acceptable salt thereof, is in the range of about 1 to 20 μm, more preferably in the range of about 2 to 10 μm.

15. The rapidly disintegrating tablet according to claim 1, wherein the rapidly disintegrating tablet is manufactured by the direct compression method.

16. The rapidly disintegrating tablet according to claim 1, wherein the rapidly disintegrating tablet is disintegrated in the buccal cavity upon contact with saliva in less than 1 minute, preferably in less than 40 seconds.

17. The rapidly disintegrating tablet according to claim 1, wherein 85% of the rosuvastatin or pharmaceutically acceptable salts thereof, is released within 30 minutes, preferably more than 90% is released within 20 minutes.

18. The rapidly disintegrating tablet suitable for oral administration comprising;
a) 1% w/w to about 8% w/w, preferably in the range from about 2% w/w to about 7% w/w of rosuvastatin or pharmaceutically acceptable salts thereof;
b) 0.5% w/w to about 10% w/w, preferably in the range of about 1% w/w to about 7.5% w/w of Croscarmellose sodium;
c) a combination of 10% w/w to about 90% w/w, preferably in the range of about 15% w/w to about 85% w/w of microcrystalline cellulose and mannitol; and
e) one or more pharmaceutical acceptable excipient.
